(19) United States
(12) Patent Application Publication
(10) Pub. No.: US 2012/0109046 A1
(43) Pub. Date: May 3, 2012

(54) SOLID DOSE DELIVERY DEVICE

(75) Inventors: Henrik Ljunggreen, Ballerup (DK); Torben Strom Hansen, Lyngby (DK)

(73) Assignee: Novo Nordisk A/S, Bagsvaerd (DK)

(21) Appl. No.: 13/257,834

(22) PCT Filed: Mar. 26, 2010
(86) PCT No.: PCT/EP2010/053989

§ 371 Date: Dec. 13, 2011

Related U.S. Application Data
(60) Provisional application No. 61/165,148, filed on Mar. 31, 2009.

(30) Foreign Application Priority Data
Mar. 27, 2009 (EP) 09156458.3

(51) Int. Cl. A61M 5/31

(52) U.S. Cl. 604/60; 604/59

(57) ABSTRACT

The invention relates to a medicament delivery device (1, 100) for delivering one or more solid dose pegs (10, 110) through the skin. The device (1, 100) has a number of exit openings (9, 109) corresponding to the number of solid dose pegs (10, 110) to be delivered. A detachable portion (3, 103) of the device (1, 100) is removed before use to uncover the exit openings (9, 109) and a release rod (17, 117). When the exit openings (9, 109) are pressed against the skin the release rod (17, 117) automatically disables a safety mechanism adapted to prevent unintended activation of the device (1, 100).

### FIELD OF THE INVENTION

The present invention relates generally to a device for delivering a medicament, such as glucagon or insulin, to a subject. More specifically, the invention relates to a device for delivering a dose of a solid medicament.

### BACKGROUND OF THE INVENTION

Therapeutic compositions in solid dose form may be used for delivery of bioactive materials to various tissue types, including intradermal and subcutaneous tissues. One way of enabling intra- or hypodermal administration of a solid dose drug is to prepare the drug in the shape of a pointed projectile or peg, and load it into an injection device which is capable of driving it through the epidermis. Advantages of such solid dose administration compared to administration by hypodermic injections of a liquid material are e.g. a reduced risk of infection and elimination of needle stick injuries. Further, people with needle-phobia, or people who simply do not like the thought of inserting a needle through the skin, may view solid dose delivery as an attractive alternative to a needle-based treatment option. Also in relation to storage a solid dose delivery device may be advantageous as most medicines have a longer shelf life in a solid state than in a solution, and drugs in solid form are generally less susceptible to chemical degradation in a changing thermodynamical environment.

U.S. Pat. No. 6,102,896 discloses various manually operated mechanisms for insertion of a medication containing dissolvable glass needle. Different delivery device configurations are shown, all appearing oblong or rather bulky. U.S. Pat. No. 7,470,250 discloses a small apparatus for automatic insertion of a medicine peg into the skin using a flexible plunger. Both documents concern delivery devices for the insertion of a single solid dose peg containing the entire prescribed dose.

U.S. Pat. No. 4,846,793 discloses a device for subcutaneous implantation of a plurality of solid medicinal pellets. The pellets are arranged in line in a hollow needle and are adapted to be ejected therethrough one after the other by means of an obturator. The needle is used to penetrate the skin and to locate the desired position in the skin for the pellets to be placed. During implantation of the pellets the needle is moved back and forth in the skin to ensure proper positioning.

### SUMMARY OF THE INVENTION

Even though the prior art solid dose delivery devices may be relatively small they are of configurations which are not particularly appealing to a person which has to carry such a device around at all times. This may result in the undesirable situation that the person sometimes chooses to leave the device at home for convenience reasons. Within certain therapy areas convenience and easy dose access are of high importance in relation to improving treatment compliance and increasing the general safety of the patient. Hence, it is relevant that the particular device design addresses issues such as portability and integration with the daily routines of the users. Further, in relation to solid dose delivery, when inserting solid dose pegs of a certain dose size it may be advantageous to divide the dose into a number of smaller pegs and insert these pegs separately in order to obtain a faster absorption and to avoid an uncomfortably large implant being inserted at one site.

Having regard to the above, it is an object of the invention to provide a small, handy, easy-to-carry and easy-to-use solid dose delivery device which has a size and a configuration that do not tempt potential users to leave it at home instead of bringing it along during the day.

It is a further object of the invention to devise a solid dose delivery device capable of delivering a plurality of solid dose implants in a manner which does not involve the use of a needle and which requires a minimum number of operating steps.

It is an even further object of the invention to provide a substance delivery device which is secured against unintentional operation and which can be readied for delivery quickly.

In the disclosure of the present invention, aspects and embodiments will be described which will address one or more of the above objects or which will address objects apparent from the below disclosure as well as from the description of exemplary embodiments.

Thus, in a first aspect of the invention a solid dose delivery device is provided, comprising: a thin, credit card like housing, a solid dose peg, an exit opening for the solid dose peg, ejection means for ejecting the solid dose peg from the delivery device through the exit opening, and activation means for causing activation of the ejection means.

ISO 7810 defines the ID-1 format which is commonly used for banking cards such as credit cards. This format specifies a structure having a longitudinal dimension of 85.6 mm and a transversal dimension of 53.98 mm. ISO 7813 defines additional characteristics of ID-1 banking cards, for example a height of 0.76 mm. In the present context the term “credit card like housing” should be interpreted as meaning a housing of credit card like configuration, e.g. a slim structure capable of being received in a compartment dedicated to hold one or more credit cards. The structure may have a rectangular base with rounded corners, much like a conventional credit card. However, it is appreciated that other structures may just as well be implemented within the scope of the invention, e.g. structures having a strictly rectangular base, a quadratic or pentagonal base with or without rounded corners, an oval or elliptical base, or other structures capable of being substantially received in a dedicated credit card compartment, including structures having a varying height as well as a varying longitudinal and/or transversal dimension. Preferably, at least 80% of the structure has a height of 10 mm or less, more preferably of 8 mm or less, even more preferably of 6 mm or less and most preferably of 4 mm or less. Further, preferably, the largest longitudinal dimension of the structure base is 100 mm or less, more preferably 90 mm or less, and most preferably 85.6 mm or less. Even further, preferably, the largest transversal dimension of the structure base is 70 mm or less, more preferably 60 mm or less, and most preferably 54 mm or less. The maximum height of the structure may specifically be 10 mm. Alternatively, the maximum height of the structure is 8 mm, or 6 mm, or 4 mm.

In the present context, a “solid dose peg” should be interpreted as any kind of cohesive solid medicament of a shape which allows it to be introduced into the body through the skin without the aid of a penetrative tool, e.g. a solid medicament shaped as a pointed projectile.

It may be advantageous to divide the dose size into two or more solid dose pegs and to insert these at different sites in order to increase the dissolution rate of the medicament in the skin and to minimise any discomfort associated with delivering the entire dose at a single site. Furthermore, multiple insertions enable a wider distribution of the dose in the skin. Therefore, the solid dose delivery device may comprise more than one solid dose peg and more than one exit opening, the number of exit openings preferably corresponding to the number of solid dose pegs.

The ejection means may comprise a moveable rod, which moveable rod may be arranged to move in the delivery device in conjunction with a carriage or a slider along one or more guiding means. In case the delivery device comprises more than one solid dose peg and more than one exit opening, the ejection means may comprise a corresponding number of moveable rods, each moveable rod being adapted to eject a dedicated solid dose peg from the delivery device through its corresponding exit opening. In an exemplary embodiment, the more than one solid dose pegs are ejected from the delivery device substantially simultaneously, e.g. by activating each moveable rod at substantially the same time, or by providing a physical interrelation between the moveable rods which prevents them from moving individually. Such a physical interrelation could e.g. be a common transportation member, such as a carriage, capable of guided movement in the housing. The ejection means may alternatively comprise one or more springs adapted to impact the one or more solid dose pegs, or other means capable of driving the solid dose pegs out of the housing.

The extent of movement of the one or more moveable rods may be restricted in accordance with the desired depth of insertion of the solid dose pegs in the skin. If e.g. the delivery device contains a medicament which is recommended for subcutaneous administration the delivery device may be constructed such that the one or more moveable rods will protrude some millimetres from the respective exit openings after ejection of the one or more solid dose pegs. Otherwise, the delivery device may e.g. be constructed such that the one or more moveable rods do not protrude from the housing at any time.

The ejection means may be activated via activation means to eject the solid dose through the skin entirely manually. In that case the one or more moveable rods are operable from outside the delivery device, e.g. by a slidable pin. Alternatively, the activation means may comprise an energy source, such as a spring member, e.g. a leaf spring or a coil spring, or a small explosive charge, which the user can activate at a desired time to perform an automatic ejection of the one or more solid dose pegs.

In a particular embodiment the solid dose delivery device comprises specifically two solid dose pegs and two exit openings, whereby the physical size of the implant to be inserted through the skin is halved. This causes a smaller incision at the respective insertion sites compared to the incision caused by a single solid dose peg containing the whole dose and provides for a faster dissolution of the medicament in the skin, and thereby a faster uptake of the active agent in the circulatory system, while entailing an acceptable drawback of having to tolerate two insertions instead of one.

The two solid dose pegs may either be ejected from the delivery device simultaneously, or substantially simultaneously, or successively. If they are ejected simultaneously, or substantially simultaneously, the user will only feel a penetration of the skin once. This may be preferred by people who are reluctant to perform injections of any kind.

A portion of the housing may be adapted to be detached from the rest of the housing to uncover the one or more exit openings before use. Thereby, a sterile environment for the medicament is preserved until right before administration, and the delivery device can be readied by a single, simple operation involving only the detachment of a housing portion. The detachable portion may be an originally integrated portion of the housing, or it may be a detachably mounted part, like e.g. a cap. In case the detachable portion is an integrated portion of the housing, the delivery device may be provided with a weakened section and/or other aids, e.g. a perforated line along which the detachable portion can be torn, allowing for easy detachment of the detachable portion. In a particular embodiment, detaching the detachable housing portion from the rest of the housing uncovers the one or more exit openings and a contact face adapted to bear against a portion of the user's skin during delivery of the solid dose drug. Alternatively, or additionally, detaching the detachable housing portion from the rest of the housing uncovers one or more peel-off foils or penetrable membranes adapted to provide an anti-bacterial sealing of the one or more exit openings until activation of the ejection means.

The delivery device is intended to be carried around by a user during the entire day in a way that causes a minimum of nuisance. Especially if the delivery device contains a medicament which the user irregularly, or maybe even rarely, is in need of, she/he may be tempted to leave it at home if it has a size that makes it problematic to carry around without a handbag or the like. However, some irregularly or rarely needed medicaments are of vital importance to the specific user, and it may be fatal for her/him to not have immediate access to that particular medicament. For example, a person with diabetes may suddenly need glucagon to counteract e.g. an overdose of insulin or too much physical stress causing a hypoglycaemic event. If the delivery device is of a credit card like configuration, the user can carry it around in e.g. a purse or a pocket at all times without having to pay further attention to it. Since most purses include special pockets for credit cards and a general credit card like configuration does not take up much space in a jacket or a pair of trousers it will thus be easier to establish a habit amongst users of always bringing the respective delivery device along with them when leaving home, and to thereby ensure greater safety for these users.

When carrying such a delivery device around during the day there is a risk that the medicament is ejected accidentally. In a particular embodiment the solid dose delivery device further comprises safety means for preventing unintended operation of the activation means. The safety means may comprise a portion of the housing being detachable from the rest of the housing. In that case detaching the detachable housing portion from the rest of the housing may uncover means for releasing a locking means. This prevents the delivery device from being subjected to any dose related activation prior to removal of the detachable portion. In a particular embodiment detaching the detachable portion of the housing from the rest of the housing uncovers the means for releasing the locking means, as well as the one or more exit openings and the contact face adapted to bear against a skin portion. Thereby, the delivery device can be readied by two simple operations involving only the detachment of a housing portion and the release of the locking means.

The safety means may alternatively, or additionally, comprise locking means which prevents the ejection means from performing an ejection of the solid dose medicament, either directly, through a locking engagement with the ejection means, or indirectly, through a locking engagement with the activation means, e.g. with a slidable pin or an activation button. Releasing means for releasing the locking means may be operable from outside the delivery device and this means may comprise a release member being either in engagement with the ejection means, or coupled to a locking member, which is in engagement with the ejection means or with the activation means. The releasing means may be adapted to move in the housing in such a way that when moved in a specific direction the releasing means will cause the locking member to move out of engagement with e.g. the activation means, whereby the activation means, e.g. the slidable pin or the activation button, can be operated to activate the ejection means. A portion of the releasing means may protrude from the housing, e.g. after removal of the detachable portion, in such a way that when the contact face and/or the exit openings are pressed against the skin at least a portion of the portion of the releasing means protruding from the housing is pushed into the housing in a movement that causes a disabling of the locking means. When the user finds that the delivery device is properly positioned on the skin she/he then simply slides the slidable pin or presses the activation button to inject the solid dose medicament.

The locking means may be biased against the releasing means, or the releasing means may otherwise be biased, such that if the user for some reason chooses to postpone a prepared administration of the solid dose medicament and leaves the delivery device temporarily on e.g. a table the releasing means will be returned to the original position in which it protrudes from the housing and in which the locking means blocks the activation means. The delivery device is thereby secured against activation until the releasing means is once again pressed into the housing. The releasing means may be provided with means for restricting its movements within the housing. The locking means may comprise a structure being rotatably arranged in such a way that a portion of the structure engages with, or abuts, the activation means, e.g. the slidable pin or the activation button, in certain angular positions, thereby preventing movement of the slidable pin or the activation button, and is moved out of engagement, or contact, with the slidable pin or the activation button in other angular positions, thereby allowing movement of the slidable pin or the activation button. Alternatively, the locking means may comprise a tiltable pin or another suitable structure capable of being moved into and out of engagement with the activation means. The releasing means may comprise a rod or a pin, or indeed any structure capable of transferring a force, or a moment of force, to the locking means to alter its position.

The ejection means may be arranged diagonally, or somewhat diagonally, in the delivery device. Alternatively, the ejection means may be arranged parallel to one of the housing sides. In either case, there may be room left in the housing for various accessories relevant for the treatment or for other purposes. The housing may therefore be adapted to also contain e.g. a pair of scissors and/or a pair of tweezers. Such tools may be useful in case an activation of the ejection means for some reason causes an incomplete insertion and a solid dose peg must be removed from the skin.

Due to the self-contained credit card like design, which is capable of preserving an anti-bacterial environment for the medicament until use, the delivery device does not need further packaging and can be supplied to the user with the instructions placed on one or more of its surfaces.

In a second aspect of the invention a solid dose delivery device is provided, comprising: a housing, a solid dose peg, an exit opening for the solid dose peg, ejection means for ejecting the solid dose peg from the delivery device through the exit opening, and activation means for causing activation of the ejection means. The solid dose delivery device may further comprise safety means for preventing unintended operation of the activation means.

The safety means are adapted to function as means for preventing, or at least limiting the risk of, premature ejection of the solid dose peg from the delivery device and may comprise features as described above in relation to the first aspect of the invention, particularly with respect to the locking means and the releasing means.

The housing may be flat, pen-shaped, box-shaped, or have any other appearance relevant for a portable medical device. Preferably, the solid dose delivery device comprises a contact face adapted to bear against a portion of the user's skin during ejection of the solid medicament, and the exit opening may be arranged in the contact face such that when the user wants to administer the solid dose she/he simply places the contact face against the skin and operates the activation means. In case the solid dose delivery device comprises the safety means, placing the contact face against the skin may cause the releasing means to automatically release the locking means, e.g. as described above in relation to the first aspect of the invention.

The housing may comprise a detachable, e.g. originally integrated, portion adapted to be detached from the rest of the housing before use of the delivery device. Detaching the detachable portion of the housing from the rest of the housing may uncover the contact face, the exit opening and/or the releasing means, e.g. as described above in relation to the first aspect of the invention. The housing may be provided with a weakened section in the area from which the detachable portion can be detached. The weakened section may comprise perforations or other suitable means allowing for easy detachment of the detachable housing portion, such as by tearing off, breaking off, twisting off or otherwise removing the housing portion.

In a third aspect of the invention a solid dose delivery device is provided, comprising: a housing, a solid dose peg, an exit opening for the solid dose peg, ejection means for ejecting the solid dose peg from the delivery device through the exit opening, and activation means for causing activation of the ejection means, wherein a portion of the housing is detachable from the rest of the housing. The detachable housing portion is adapted to be removed from the delivery device before use. The detachable portion may be an originally integrated portion of the housing or a detachably mounted part, and it preferably covers at least the exit opening prior to detachment. The solid dose delivery device may comprise a contact face as described above in relation to the first and second aspects of the invention, and the detachable housing portion preferably also covers at least a portion of the contact face prior to detachment.

The housing may be flat, pen-shaped, box-shaped, or have any other appearance relevant for a portable medical device, and it may be provided with a weakened section in the area from which the detachable portion can be detached. The weakened section may comprise perforations or other suitable means allowing for easy detachment of the detachable housing portion, such as by tearing off, breaking off, twisting off or otherwise removing the housing portion.

Safety means, comprising locking means and releasing means, may be provided for preventing unintended operation of the activation means, and may comprise features as described above in relation to the first and second aspects of the invention. In that case the detachable portion of the housing may comprise a cavity adapted to occupy at least a portion of the releasing means when the detachable portion is attached to, or still a portion of, the housing, e.g. as supplied by the manufacturer. Thereby, upon removal of the detachable housing portion from the rest of the housing, the at least a portion of the releasing means will be available for operation.

In a fourth aspect of the invention a solid dose delivery device is provided, comprising: a housing, two solid dose pegs, an exit opening for each of the solid dose pegs, ejection means for ejecting the solid dose pegs from the delivery device through the exit openings, and activation means for causing activation of the ejection means. In an exemplary embodiment, the ejection means are adapted to eject the solid dose pegs simultaneously, or substantially simultaneously, whereby the entire dose is delivered to the user following only one operation of the activation means.

The ejection means may comprise two force transmitting members, e.g. two rods, two springs or other objects capable of transmitting a driving force, for impacting the solid dose pegs and forcing them through the respective exit openings. These force transmitting members may be arranged separately or they may be coupled, e.g. via a transportation member adapted to move in the housing, such that activation of the ejection means causes a simultaneous, or substantially simultaneous, movement of the force transmitting members. In an exemplary embodiment the force transmitting members are arranged in parallel and adjusted such that upon activation of the ejection means the force transmitting members will impact the respective solid dose pegs simultaneously, or substantially simultaneously.

Safety means, comprising locking means and releasing means, may be provided for preventing unintended operation of the activation means, and may comprise features as described above in relation to the other aspects of the invention. In that case the locking means may be adapted to prevent movement of the force transmitting members, e.g. through a releasable locking engagement with the transportation member.

The solid dose delivery device may comprise more than two solid dose pegs and more than two exit openings, the number of exit openings preferably corresponding to the number of solid dose pegs, and the ejection means may comprise a number of force transmitting members which corresponds to the number of solid dose pegs.

It is noted that even though the above described aspects and embodiments of the invention concern a solid dose delivery device, i.e. a delivery device adapted to deliver one or more doses of a solid substance, the principles of the invention are just as applicable to e.g. a jet injection device, i.e. a delivery device adapted to inject a liquid substance through the skin of a subject by means of a high pressure jet.

It is emphasized that a skilled person would readily recognise that any feature described in relation to a specific aspect of the present invention could also be combined with any of the other aspects of the invention as set out in the above.

In the present specification reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in at least that one aspect or embodiment of the invention, but not necessarily in all aspects or embodiments of the invention. It is emphasized, however, that any combination of features, structures and/or characteristics described in relation to the various aspects and embodiments of the invention is encompassed by the invention unless otherwise indicated herein or clearly contradicted by context.

### BRIEF DESCRIPTION OF THE DRAWINGS

In the following the invention will be further described with references to the drawings, wherein

FIG. 1 shows a top view of a solid dose delivery device according to an embodiment of the invention,

FIG. 2 shows a side view of the solid dose delivery device of FIG. 1, and

FIG. 3 shows a cross-sectional view of a solid dose delivery device according to another embodiment of the invention.

In the figures like structures are mainly identified by like reference numerals.

### DESCRIPTION OF EXEMPLARY EMBODIMENTS

When in the following relative expressions, such as “clockwise” and “counter-clockwise”, are used, these refer to the appended figures and not necessarily to an actual situation of use. The shown figures are schematic representations for which reason the configuration of the different structures as well as their relative dimensions are intended to serve illustrative purposes only.

The present invention is directed to small and easy-to-use devices for delivery of a solid medicament, particularly for such devices being of a configuration which allows for convenient transportation, such as e.g. in a credit card pocket of a purse or a dedicated credit card holder.

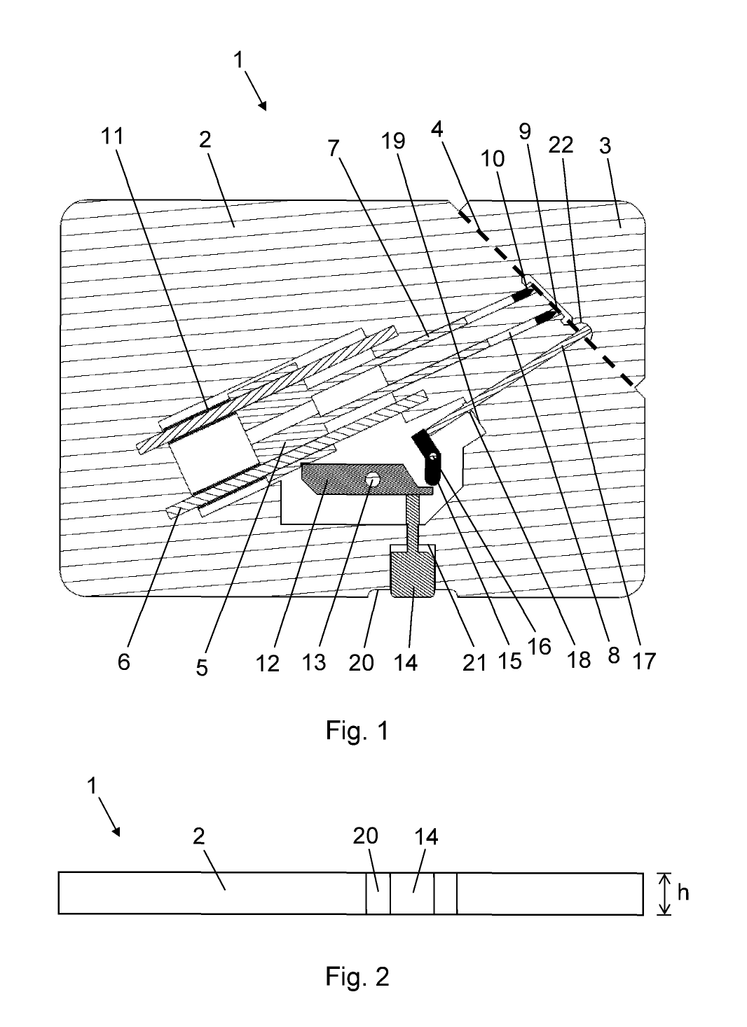

FIG. 1 shows a delivery device 1 for delivering a dose of a medicament in the form of two solid pegs 10. The delivery device 1 has a housing 2 of a generally rectangular plane shape and a height which allows it to be easily fitted into a purse or a pocket. The delivery device 1 is in size comparable with a credit card. It measures approximately 85 mm×55 mm in the largest plane and has a height of about 3-4 mm. In FIG. 1 the delivery device 1 is shown in a storage or non-use situation, as supplied by the manufacturer.

The housing 2 is provided with a weakened section 4 enabling a detachable portion 3 of the housing 2 to be readily torn off before use of the delivery device 1. The housing 2 holds a slider 5 slidably arranged on a couple of guiding rails 6. The slider 5 has two parallel extensions 7, each extension 7 being slidably occupied in a barrel 8 and being adapted to eject a solid peg 10, also arranged in the barrel 8, through an opening 9. A couple of springs 11 are provided for energising the slider 5 to slide along the guiding rails 6 in a direction towards the solid pegs 10.

In FIG. 1 the slider 5 is in a retracted position ready to be fired, and both springs 11 are compressed. The slider 5 is retained in this position by a locking pin 12. The locking pin 12 is rotatable about a swivel 13 and is biased by a spring (not shown) to rotate clockwise about the swivel 13. The locking pin 12 is thereby biased against both the slider 5 and an activation button 14. A bead on the activation button 14 defines the limit of outward movement of the activation button 14 relative to the housing 2. A bracket 15 is able to rotate about a hinge 16, and is biased by a spring (not shown) to rotate in a specified direction, corresponding to a clockwise direction in FIG. 1. A release rod 17 is coupled to the bracket 15 and is capable of rotating the bracket 15 counter-clockwise after removal of the detachable portion 3 from the housing 2. A bead 18 on the release rod 17 abuts an inner wall 19 of the housing 2, whereby the release rod 17 sustains the biasing moment of rotation from the spring (not shown) acting on the bracket 15. Until the detachable portion 3 is separated from the housing 2 the release rod 17 is inaccessible from the outside. A portion of the release rod 17 is occupied in a recess 22 in the detachable portion 3 and exposed for operation only upon removal of the detachable portion 3 from the housing 2. This provides a safety against unintended activation of the delivery device 1 during e.g. storage and transport.

The position of the bracket 15 in the shown non-use situation prevents the activation button 14 from being depressed to release the slider 5 because the locking pin 12 is prevented from rotating about the swivel 13. The activation button 14 is arranged in a recess 20 in the housing 2 and is, when the bracket 15 is rotated away from its storage position by the release rod 17 during use of the delivery device 1, capable of being depressed to a stop 21. Depressing the activation button 14 causes the locking pin 12 to rotate counter-clockwise about the swivel 13, whereby the locking pin 12 is brought out of engagement with the slider 5. The slider 5 is then able to slide along the guiding rails 6 under the force of the springs 11.

FIG. 2 shows a side view of the delivery device 1, having a uniform height, h. This enables an easy fitting of the delivery device 1 in e.g. a pocket or a flexible credit card receiving compartment.

In a situation of use, a person in need of a dose of the medicament, or a person helping the person in need of the medicament, takes the delivery device 1 and tears off the detachable portion 3 to uncover the openings 9 as well as the release rod 17. The thereby uncovered portion of the housing 2, as indicated by the weakened section 4, is then pressed against the skin (not shown) at a desired site of injection. As long as the delivery device 1 is not properly positioned relative to the skin the activation button 14 is unable to move in the recess 20, and the slider 5 is secured against activation due to the locking pin 12 being held in locking position, as described in the above.

When the delivery device 1 is properly positioned on the skin the normal force acting between the skin and the release rod 17 will cause the release rod 17 to move linearly in the housing 2, thereby rotating the bracket 15 counter-clockwise about the hinge 16 and out of engagement with the locking pin 12. This disengagement enables the activation button to be depressed in the recess 20. The user then depresses the activation button 14 to the stop 21, which depression will cause the locking pin 12 to rotate counter-clockwise about the swivel 13 and thereby to move out of engagement with the slider 5. As the locking pin 12 moves out of engagement with the slider 5 the springs 11 release energy for fast forward motion of the slider 5 towards the openings 9. The extent to which the slider 5 is able to advance so is defined by an inner geometry of the housing 2. In this embodiment the slider 5 advances just enough to allow the extensions 7 to impact the solid pegs 10 and eject them through the openings 9 substantially simultaneously. The solid pegs 10 are each provided with a pointed tip to enable uncomplicated penetration of the skin. In other embodiments the slider 5 is able to advance a little further to allow the extensions 7 to pass some millimetres into the skin to ensure e.g. a subcutaneous insertion of the solid pegs 10. After insertion of the solid pegs 10 the delivery device 1 can be discarded.

The embodiment of FIG. 1 depicts the detachable portion 3 as a corner portion of the delivery device 1 and the guiding rails as arranged somewhat diagonally in the housing 2. However, it is clear that the detachable portion 3 can just as well be detachable along a line parallel to one of the sides of the delivery device 1, and the guiding rails 11 can be arranged in various other ways, such as substantially perpendicularly to the current detachment line. Further, in some embodiments of the invention a different number of springs 11 may be employed to advance the slider 5, e.g. a single spring or more than two springs, just as a different number of extensions 7 may be provided on the slider 5 and arranged in respective barrels 8 to eject a corresponding number of solid pegs 10 through respective openings 9.

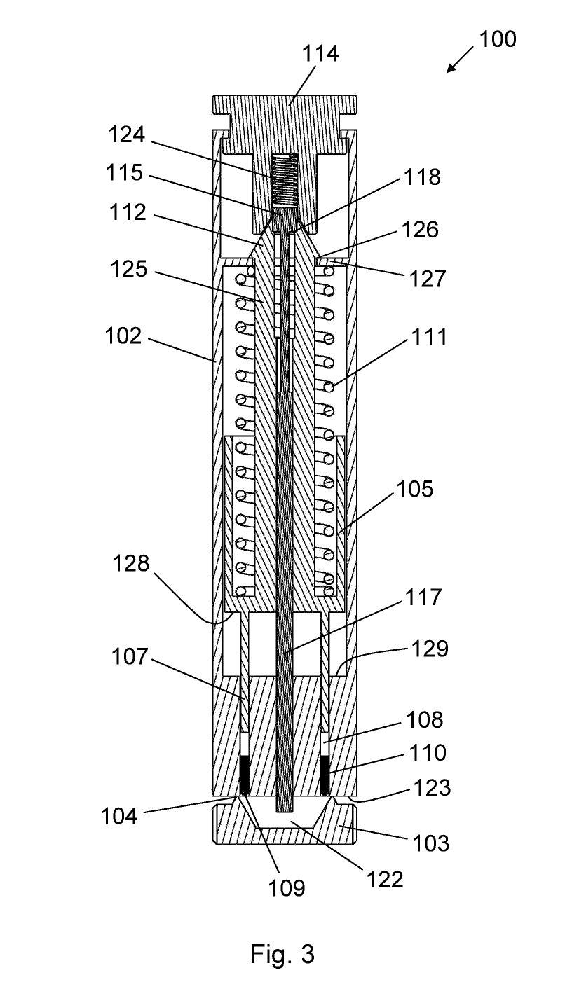

FIG. 3 shows a cross-sectional view of a delivery device 100 according to another exemplary embodiment of the invention. The delivery device 100 has a housing 102 of cylindrical form, much alike that of a so-called pen injector. Attached to an abutment surface 123 of the housing 102, e.g. by welding or gluing, is a tear-off portion 103 which covers a distal end portion of a release rod 117 and two exit openings 109 for respective solid pegs 110, the tear-off portion 103 thereby providing a sterile barrier for the medicament as a part of the primary packaging. The tear-off portion 103 is easily detachable from the housing 102 by breakage of a weakened section 104. Before such detachment the tear-off portion 103 together with a portion of the abutment surface 123 define a recess 122 for the distal end portion of the release rod 117. Thereby, it is ensured that the release rod 117 is inaccessible to a user when the delivery device 100 is in a storage condition, this constituting a safety feature, since inadvertent operation of the release rod 117 is prevented.

The solid pegs 110 are housed in respective barrels 108, and the release rod 117 is slidably arranged in a central portion of the housing 102. Also arranged in the housing 102 is a slider 105 which is adapted to be moved from an initial position (shown in FIG. 3) to a fired position following release of a spring 111. When in the fired position a front face 128 of the slider 105 abuts a stop wall 129 of the housing 102. The slider 105 has a couple of extension rods 107 which during movement of the slider 105 to the fired position will impact the solid pegs 110 and eject them through the exit openings 109. The solid pegs 110 are sharpened to enable easy penetration of the subject's skin when thus ejected from the housing 102.

Before use the spring 111 is compressed between the slider 105 and a flange 127 in the housing 102. A couple of flexible arms 125 associated with the slider 105 extend proximally beyond the flange 127, each flexible arm 125 terminating in a partial spearhead 112 having a catch portion 126 which is held in place against the flange 127 by the downward pushing force on the slider 105 from the spring 111.

A push button 114 extends out of the proximal end portion of the housing 102 and functions as an injection activator. A spring 124 is arranged in connection with the push button 114 and is compressed between the push button 114 and an enlarged head 115 on the release rod 117. The spring 124 thereby biases the enlarged head 115 against protrusions 118 on the partial spearheads 112, providing a limitation of longitudinal downward movement of the release rod 117.

As long as the tear-off portion 103 is attached to the housing 102 the push button 114 is prevented from being depressed. This is because of the spring 124 biased position of the enlarged head 115 between the two partial spearheads 112 preventing any radial inward movement of the flexible arms 125. If the flexible arms 125 are unable to deflect inwardly towards the central axis of the housing 102 the contact between the respective catch portions 126 and the flange 127 will prevent any downward movement of the slider 105, and so the contact interface between the partial spearheads 112 and the push button 114 will prevent a downward movement of the push button 114.

In a use situation, the delivery device 100 is grabbed and the tear-off portion 103 is removed, e.g. by twisting it relative to the housing 102, to reveal the abutment surface 123. The abutment surface 123 is then pressed firmly against the skin of a subject, whereby the normal force acting between the skin and the distal end portion of the release rod 117 will overcome the biasing force of the spring 124, and the release rod 117 will be moved towards the distal, or back, end of the delivery device 100. This backward movement of the release rod 117 will compress the spring 124 further and move the enlarged head 115 out of contact with the partial spearheads 112. Now, if the user decides to temporarily postpone the administration of the drug she/he simply removes the delivery device 100 from the skin, whereby the spring 124 will force the release rod 117 back to its initial position with the enlarged head 115 resting against the protrusions 118. The user does then not have to worry about inadvertently firing the delivery device 100 because the push button 114 is again prevented from being depressed.

However, if the user maintains the position of the abutment surface 123 on the skin the push button 114 can be depressed. This will result in an inward deflection of the flexible arms 125, whereby the catch portions 126 will slide out of contact with the flange 127, and the spring 111 will be released, driving the slider 105 forward to the stop 129. The movement of the slider 105 results in the extension rods 107 pushing the solid pegs 110 through the exit openings 109 and into the skin.

### EXAMPLES

In the following different examples embodying the invention in accordance with the first aspect are presented.

### Example 1

A solid dose delivery device, comprising: a substantially credit card sized, or a credit card like, housing, a solid dose peg, an exit opening for the solid dose peg, ejection means for ejecting the solid dose peg from the delivery device through the exit opening, and activation means for causing activation of the ejection means.

### Example 2

A solid dose delivery device as in example 1, further comprising safety means for preventing unintended operation of the activation means.

### Example 3

A solid dose delivery device as in example 2, wherein the safety means comprises locking means, the locking means being releasable via releasing means.

### Example 4

A solid dose delivery device as in example 3, wherein the activation means comprises an activation button, and wherein releasing the locking means enables operation of the activation button.

### Example 5

A solid dose delivery device as in example 4, further comprising a contact face adapted to bear against a skin portion during ejection of the solid dose peg, wherein placing the contact face against the skin causes the releasing means to automatically release the locking means, thereby enabling operation of the activation button.

### Example 6

A solid dose delivery device as in example 5, wherein the releasing means comprises a release member adapted to move in the housing between an initial position in which the activation button is inoperable and a readied position in which the activation button is operable, and wherein a portion of the release member protrudes from the contact face when the release member is in the initial position.

### Example 7

A solid dose delivery device as in example 6, wherein the release member is completely occupied in the housing when the release member is in the readied position.

### Example 8

A solid dose delivery device as in example 1, wherein the housing comprises a detachable portion adapted to be detached from the rest of the housing before use of the delivery device.

### Example 9

A solid dose delivery device as in example 8, further comprising a contact face adapted to bear against a skin portion during ejection of the solid dose peg, wherein the detachable portion covers the contact face before detachment.

### Example 10

A solid dose delivery device as in example 8 or 9, wherein the detachable portion covers the exit opening before detachment.

### Example 11

A solid dose delivery device as in any of examples 8-10, wherein the detachable portion is an integrated portion of the housing.

### Example 12

A solid dose delivery device as in any of examples 8-11, further comprising safety means for preventing unintended operation of the activation means.

### Example 13

A solid dose delivery device as in example 12, wherein the safety means comprises locking means, the locking means being releasable via releasing means, and wherein the releasing means is operable only after detachment of the detachable portion.

### Example 14

A solid dose delivery device as in example 13, wherein the detachable portion covers a portion of the releasing means before detachment.

### Example 15

A solid dose delivery device as in any of the preceding examples, comprising more than one solid dose peg and more than one exit opening, wherein the solid dose pegs are ejected from the delivery device through the exit openings substantially simultaneously.

### Example 16

A solid dose delivery device as in any of the preceding examples, wherein the activation means comprises an energy source, and wherein the ejection means operates in response to the energy source releasing energy.

### Example 17

A solid dose delivery device as in example 16, wherein the energy source comprises a spring member.

In the following different examples embodying the invention in accordance with the second aspect are presented.

### Example 18

A substance delivery device, comprising: a housing, a volume of substance to be delivered, an exit opening for the volume of substance, ejection means for ejecting the volume of substance from the delivery device through the exit opening, activation means for causing activation of the ejection means, and safety means for preventing unintended operation of the activation means.

### Example 19

A substance delivery device as in example 18, wherein the substance delivery device is a solid dose delivery device and the volume of substance is a solid dose peg.

### Example 20

A substance delivery device as in example 18, wherein the substance delivery device is a jet injection device, and the volume of substance is a dose of liquid drug.

### Example 21

A substance delivery device as in any of examples 18-20, wherein the safety means comprises locking means, the locking means being releasable via releasing means.

### Example 22

A substance delivery device as in example 21, wherein the activation means comprises an activation button, and wherein releasing the locking means enables operation of the activation button.

### Example 23

A substance delivery device as in example 21 or 22, further comprising a contact face adapted to bear against a skin surface, and wherein placing the contact face against the skin causes the releasing means to automatically release the locking means.

### Example 24

A substance delivery device as in example 23, wherein the exit opening is arranged in the contact face.

### Example 25

A substance delivery device as in any of examples 21-24, wherein at least a portion of the releasing means projects from the housing.

### Example 26

A substance delivery device as in example 25, wherein the at least a portion of the releasing means projects from the contact face.

### Example 27

A substance delivery device as in any of examples 21-26, wherein the releasing means is adapted to move in the housing.

### Example 28

A substance delivery device as in example 21 or 22, further comprising a contact face adapted to bear against a skin surface during ejection of the substance, wherein the exit opening is arranged in the contact face, at least a portion of the releasing means projects from the contact face, and the releasing means is moveably arranged in the housing, and wherein placing the contact face against the skin causes at least a portion of the portion of the releasing means that projects from the contact face to be pressed into the housing, thereby releasing the locking means.

### Example 29

A substance delivery device as in any of examples 21-28, wherein the housing comprises a detachable portion adapted to be detached from the rest of the housing before use of the delivery device.

### Example 30

A substance delivery device as in example 29, wherein the detachable portion covers the exit opening and/or a portion of the releasing means before detachment.

### Example 31

A substance delivery device as in any of examples 18-30, wherein the housing is of credit card like configuration.

In the following different examples embodying the invention in accordance with the third aspect are presented.

### Example 32

A substance delivery device, comprising: a housing, a volume of substance to be delivered, an exit opening for the volume of substance, ejection means for ejecting the volume of substance from the delivery device through the exit opening, and activation means for causing activation of the ejection means, wherein the housing comprises a detachable portion adapted to be detached from the rest of the housing before use of the delivery device.

### Example 33

A substance delivery device as in example 32, wherein the substance delivery device is a solid dose delivery device and the volume of substance is a solid dose peg.

### Example 34

A substance delivery device as in example 32, wherein the substance delivery device is a jet injection device, and the volume of substance is a dose of liquid drug.

### Example 35

A substance delivery device as in any of examples 32-34, wherein the detachable portion is an integrated portion of the housing.

### Example 36

A substance delivery device as in any of examples 32-35, wherein the detachable portion covers the exit opening before detachment.

### Example 37

A substance delivery device as in any of examples 32-36, further comprising safety means for preventing unintended operation of the activation means, wherein the safety means comprises locking means, the locking means being releasable via releasing means.

### Example 38

A substance delivery device as in example 37, wherein the detachable portion covers a portion of the releasing means before detachment.

### Example 39

A substance delivery device as in example 38, wherein the detachable portion comprises a cavity adapted to occupy at least a portion of the releasing means before detachment of the detachable portion.

### Example 40

A substance delivery device as in any of examples 32-39, further comprising a contact face adapted to bear against a skin portion during ejection of the substance, wherein the detachable portion covers the contact face before detachment.

### Example 41

A substance delivery device as in example 40, wherein the exit opening is arranged in the contact face.

### Example 42

A substance delivery device as in any of examples 32-41, wherein the housing is of credit card like configuration.

In the following different examples embodying the invention in accordance with the fourth aspect are presented.

### Example 43

A solid dose delivery device, comprising: a housing, two solid dose pegs, an exit opening for each of the solid dose pegs, ejection means for ejecting the solid dose pegs from the delivery device through the exit openings, and activation means for causing activation of the ejection means, wherein the solid dose pegs are ejected from the delivery device through the exit openings simultaneously or substantially simultaneously.

### Example 44

A solid dose delivery device as in example 43, wherein the ejection means comprises two force transmitting members, each force transmitting member being adapted to impact a respective solid dose peg.

### Example 45

A solid dose delivery device as in example 44, wherein the force transmitting members are coupled so that activation of the ejection means causes a simultaneous, or substantially simultaneous, movement of the force transmitting members.

### Example 46

A solid dose delivery device as in example 45, wherein the ejection means further comprises a transportation member, and wherein the force transmitting members are coupled to the transportation member.

### Example 47

A solid dose delivery device as in example 45, wherein the ejection means further comprises a transportation member, and wherein the force transmitting members are integrated portions of the transportation member

### Example 48

A solid dose delivery device as in example 46 or 47, wherein the force transmitting members are arranged in parallel.

### Example 49

A solid dose delivery device as in any of examples 44-48, further comprising safety means for preventing unintended operation of the activation means, wherein the safety means comprises locking means, the locking means being releasable via releasing means.

### Example 50

A solid dose delivery device as in example 49, wherein the locking means prevents movement of the force transmitting members.

### Example 51

A solid dose delivery device as in any of examples 44-50, wherein each force transmitting member comprises a rod.

### Example 52

A solid dose delivery device as in any of examples 43-51, wherein the housing is of credit card like configuration.

1. A solid dose delivery device, comprising:
a housing,
a solid dose peg,
an exit opening for the solid dose peg,
ejection means for ejecting the solid dose peg from the delivery device through the exit opening, and
activation means for causing activation of the ejection means, wherein at least 80% of the housing has a height of 10 mm or less.

2. A solid dose delivery device according to claim 1, wherein at least 80% of the housing has a height of 8 mm or less.

3. A solid dose delivery device according to claim 1, wherein the housing comprises a longitudinal dimension is 100 mm or less.

4. A solid dose delivery device according to claim 1, wherein the housing comprises a transversal dimension, and the largest transversal dimension is 70 mm or less.

5. A solid dose delivery device according to claim 1, wherein the housing has a maximum height of 10 mm.

6. A solid dose delivery device according to claim 1, further comprising safety means for preventing unintended operation of the activation means, wherein the safety means further comprises locking means, the locking means being releasable via releasing means.

7. A solid dose delivery device according to claim 6, further comprising a contact face adapted to bear against a skin portion during ejection of the solid dose peg, wherein placing the contact face against the skin causes the releasing means to automatically release the locking means, thereby enabling operation of the activation means.

8. A solid dose delivery device according to claim 7, wherein the releasing means comprises a release member adapted to move in the housing between an initial position in which the activation means is inoperable and a readied position in which the activation means is operable, and wherein a portion of the release member protrudes from the contact face when the release member is in the initial position.

9. A solid dose delivery device according to claim 1, wherein the housing comprises a detachable integrated portion adapted to be detached from the rest of the housing before use of the delivery device.

10. A solid dose delivery device according to claim 9, further comprising a contact face adapted to bear against a skin portion during ejection of the solid dose peg, wherein the detachable portion covers the exit opening before detachment.

11. A solid dose delivery device according to claim 9, further comprising safety means for preventing unintended operation of the activation means.

12. A solid dose delivery device according to claim 11, wherein the safety means comprises locking means, the locking means being releasable via releasing means, and wherein the releasing means is operable only after detachment of the detachable portion.

13. A solid dose delivery device according to claim 12, wherein the detachable portion covers a portion of the releasing means before detachment.

14. A solid dose delivery device according to claim 1, comprising more than one solid dose peg and more than one exit opening, wherein the solid dose pegs are ejected from the delivery device through the respective exit openings substantially simultaneously.

15. A solid dose delivery device according to claim 1, wherein the activation means comprises an energy source.

16. A solid dose delivery device according to claim 1, wherein the ejection means operates in response to the energy source releasing energy.

17. A solid dose delivery device according to claim 1, comprising at least one of:
at least 80% of the housing has a height of 4 mm or less,
the housing comprising a longitudinal dimension of 85.6 mm or less, and
the housing comprising a largest transversal dimension of 54 mm or less.
